(19) United States
(12) Patent Application Publication
(10) Pub. No.: US 2026/0256851 A1
(43) Pub. Date: Sep. 3, 2026

(54) CARBONATED BEVERAGE COMPOSITION COMPRISING PARAXANTHINE, A MECHANISTICALLY NON-REDUNDANT DUAL CHOLINERGIC PRECURSOR SYSTEM, TAURINE-PARAXANTHINE SYNERGISTIC COMPLEX, AND A CARBONATION-STABLE SPORE- FORMING PROBIOTIC, AND METHODS OF USE THEREOF

(71) Applicants: David Michael Alexander, Brooklyn, NY (US); Michael Tolmach, Los Angeles, CA (US)

(72) Inventors: David Michael Alexander, Brooklyn, NY (US); Michael Tolmach, Los Angeles, CA (US)

(73) Assignee: Plopii LLC, Brooklyn, NY (US)

(21) Appl. No.: 19/641,518

(22) Filed: Apr. 8, 2026

(51) Int. Cl. A61K 35/742; A61K 9/00; A61K 31/198; A61K 31/375; A61K 31/4415; A61K 31/522; A61K 31/685; A61K 31/7068; A61K 31/714; A61K 35/00; A61K 36/258; A61K 47/12; A61K 47/46

(52) U.S. Cl. CPC A61K 35/742; A61K 9/0095; A61K 31/198; A61K 31/375; A61K 31/4415; A61K 31/522; A61K 31/685; A61K 31/7068; A61K 31/714; A61K 36/258; A61K 47/12; A61K 47/46; A61K 2035/11; A61K 2035/115

(57) ABSTRACT

A ready-to-drink carbonated beverage composition for cognitive enhancement comprising paraxanthine (1,7-dimethylxanthine, 100-500 mg) as an adenosine A1/A2A receptor antagonist, substantially free of caffeine; Alpha-GPC (100-600 mg) providing free choline for acetylcholine synthesis; citicoline (100-500 mg) providing cytidine for Kennedy pathway phosphatidylcholine membrane biosynthesis; taurine (500-2,000 mg) providing GABAergic modulation and osmoregulatory support; and a carbonation-stable spore-forming probiotic (Bacillus coagulans GBI-30, 6086, at least 500 million CFU) for gut-brain axis modulation. The five components simultaneously address adenosinergic, cholinergic-amplitude, cholinergic-structural, GABAergic/osmoregulatory, and gut-brain axes, producing bidirectional cognitive optimization. Preferred embodiments further comprise L-tyrosine, L-theanine, Panax ginseng extract, and vitamins in a zero-sugar 12-fluid-ounce carbonated beverage. Methods of cognitive enhancement and gut-brain axis modulation are disclosed.

### CROSS-REFERENCE TO RELATED APPLICATIONS

This application is related to co-pending U.S. patent application Ser. No. 19/630,373, filed Mar. 26, 2026, entitled “Transmucosal Oral Pouch Composition Comprising Paraxanthine and a Mechanistically Non-Redundant Dual Cholinergic Precursor System, and Methods of Use Thereof,” assigned to the same assignee as the present application, the entire disclosure of which is incorporated herein by reference. This application is also related to co-pending U.S. patent application Ser. No. 19/636,926, filed Apr. 2, 2026, entitled “Oral Tablet Composition Comprising Paraxanthine and a Mechanistically Non-Redundant Dual Cholinergic Precursor System for Headache Relief, and Methods of Use Thereof,” assigned to the same assignee, the entire disclosure of which is incorporated herein by reference.

### STATEMENT REGARDING FEDERALLY SPONSORED RESEARCH OR DEVELOPMENT

Not applicable.

### BACKGROUND OF THE INVENTION

### Field of the Invention

The present invention relates to ready-to-drink carbonated beverage compositions for cognitive enhancement, sustained mental energy, and gut-brain axis modulation, and methods of use thereof. More particularly, the invention relates to zero-sugar, zero-caffeine carbonated beverage compositions comprising paraxanthine, Alpha-glycerophosphocholine, citicoline, taurine, L-tyrosine, L-theanine, Panax ginseng extract, and a carbonation-stable spore-forming probiotic (Bacillus coagulans GBI-30, 6086, marketed as GanedenBC30®), formulated for oral ingestion as a functional energy beverage in a 12 fluid ounce (355 mL) serving format.

### I. The Functional Energy Beverage Market Gap

The global energy drink market exceeded $86 billion in 2025 (Grand View Research, 2025), yet remains overwhelmingly dependent on caffeine as the sole neuroactive stimulant. Leading products—including Red Bull®, Monster Energy®, Celsius®, and C4 Energy®—deliver caffeine at 80-300 mg per serving alongside sugar or artificial sweeteners, taurine, and B-vitamins, without meaningful cholinergic support, without addressing the well-documented limitations of caffeine pharmacology, and without gut-brain axis modulation. No commercially available energy beverage known to the inventors as of the filing date combines paraxanthine as the primary stimulant with a mechanistically non-redundant dual cholinergic precursor system (Alpha-GPC and citicoline simultaneously), a taurine-paraxanthine synergistic complex, and a carbonation-stable spore-forming probiotic in a single ready-to-drink serving.

### II. Paraxanthine as the Direct Cognitive-Active Metabolite of Caffeine

Cognitive supplements and energy beverages have historically relied on caffeine as the primary adenosine receptor antagonist. Upon CYP1A2-mediated hepatic metabolism, caffeine yields three primary methylxanthine metabolites: paraxanthine (1,7-dimethylxanthine; approximately 84% molar yield), theobromine (approximately 12%), and theophylline (approximately 4%) (Arnaud, 1985; Berthou et al., 1992). See FIG. 1. The principal cognitive benefits associated with caffeine consumption arise from paraxanthine via selective competitive antagonism of adenosine A1 and A2A receptors in the prefrontal cortex, basal ganglia, and hippocampus (Daly et al., 1993; Fredholm et al., 1999; Moran et al., 2019). Theophylline elevates heart rate and blood pressure; theobromine extends vasodilatory activity beyond the effective cognitive window (Nehlig et al., 1992). Wide interindividual CYP1A2 variance—conferred by the CYP1A2*1F allele and related polymorphisms—produces unpredictable dose-response curves, causing inconsistent cognitive benefit across the population.

Direct paraxanthine administration at 300 mg in a beverage format eliminates multi-metabolite co-exposure and circumvents CYP1A2 variability, delivering a predictable dose-response regardless of metabolizer phenotype. Paraxanthine's half-life of approximately 3.5 hours is roughly 40% shorter than caffeine's 5-7 hour half-life, materially reducing sleep disruption risk for late-day administration (Pataky et al., 2022). A 2024 randomized double-blind placebo-controlled crossover trial (PMC11089923, Texas A&M University) confirmed that paraxanthine at 200 mg outperformed an equivalent caffeine dose on post-exercise cognitive function, vigilance, and reaction time; addition of caffeine to paraxanthine conferred no statistically significant additional cognitive benefit. See FIG. 2. The 300 mg dose in the present beverage formulation provides a full-strength oral dose, accounting for gastrointestinal transit and hepatic first-pass metabolism that reduce systemic bioavailability compared to transmucosal delivery routes.

### III. The Cholinergic Gap in Energy Beverages

A critical and largely unaddressed limitation of all energy beverages—including the entirety of the commercially available energy drink market—is the complete absence of cholinergic substrate support. Acetylcholine (ACh) in the hippocampus and prefrontal cortex mediates working memory encoding, sustained attentional control, and executive function (Bartus et al., 1982; Perry et al., 1999). Sustained cholinergic firing under stimulant-driven cognitive demand progressively depletes local choline substrate and neuronal membrane phospholipid reserves, producing a post-peak cholinergic deficit not corrected by adenosine antagonism alone. No energy beverage known to the inventors provides dual cholinergic precursor support in any form.

### IV. The Dual Choline Architecture in Beverage Format

The present invention extends the Dual Choline Architecture first disclosed in the inventors' co-pending application Ser. No. 19/630,373 from the transmucosal oral pouch format to a ready-to-drink carbonated beverage at oral ingestion doses. Alpha-GPC is hydrolyzed intraneuronally by phospholipase D, releasing free choline for choline acetyltransferase (ChAT)-mediated ACh synthesis—addressing the amplitude failure mode (insufficient peak synaptic ACh concentration per firing event). Citicoline yields choline and cytidine upon systemic hydrolysis; cytidine converts to uridine in plasma, crosses the blood-brain barrier via CNT2, and enters the Kennedy pathway for phosphatidylcholine (PC) membrane biosynthesis—addressing the structural endurance failure mode (membrane degradation under sustained high-frequency cholinergic firing). See FIG. 4.

In the beverage format, Alpha-GPC is present at 300 mg and citicoline at 250 mg per 12 fl oz serving—doses calibrated for oral bioavailability after gastrointestinal transit and hepatic first-pass metabolism. These oral doses are materially higher than the transmucosal doses in the co-pending pouch application (60 mg each), reflecting the route-dependent bioavailability differential. A 2024 randomized double-blind placebo-controlled crossover trial (Kerksick, 2024, Nutrients 16 (23): 4240) demonstrated that acute Alpha-GPC administration at 630 mg significantly improved Stroop test performance (p=0.013, Cohen's d=0.61) and processing speed (p=0.021, d=0.56). The 300 mg oral dose in the present formulation provides clinically meaningful cholinergic support while preserving favorable palatability characteristics in a carbonated beverage matrix.

### V. Taurine-paraxanthine Synergistic Complex

Taurine (2-aminoethanesulfonic acid) is present at 1,000 mg per serving in the present invention. While taurine is found in certain existing energy drinks alongside caffeine, taurine has never been combined with paraxanthine in a commercial or patented formulation known to the inventors. Taurine and paraxanthine interact through mechanistically distinct and complementary pathways that produce synergistic cognitive and performance benefits not achievable by either compound alone:
GABAergic modulation: Taurine acts as an agonist at GABA-A and GABA-B receptors, providing inhibitory tone that counterbalances paraxanthine's excitatory adenosine receptor antagonism, producing smooth, sustained alertness without jitter or anxiety (El Idrissi & Trenkner, 2004; Jia et al., 2008).
Calcium homeostasis: Taurine modulates intracellular calcium signaling and maintains mitochondrial membrane potential under oxidative stress, protecting neuronal function during the elevated metabolic demand induced by paraxanthine's stimulatory effects (Schaffer et al., 2010).
Osmoregulation and cellular hydration: Taurine functions as a major organic osmolyte in neural tissue, maintaining cell volume and intracellular hydration status under conditions of elevated cognitive demand and metabolic flux (Huxtable, 1992). This osmoregulatory function supports sustained neuronal firing capacity and may reduce the subjective ‘crash’ associated with stimulant use.
Anti-inflammatory neuroprotection: Taurine inhibits NF-κB-mediated neuroinflammatory signaling and reduces microglial activation markers (Marcinkiewicz & Kontny, 2014), providing neuroprotective support complementary to but distinct from the antioxidant and adaptogenic effects of Panax ginseng.

The 1,000 mg taurine dose in combination with 300 mg paraxanthine represents a taurine-to-paraxanthine weight ratio of approximately 3.3:1 (range: approximately 2:1 to approximately 5:1 in alternative embodiments), which the inventors have identified as producing the optimal balance between GABAergic modulation and adenosinergic stimulation for a jitter-free, crash-free energy profile. See FIG. 5.

### VI. Carbonation-stable Spore-forming Probiotic and Gut-brain Axis Modulation

The present invention incorporates Bacillus coagulans GBI-30, 6086 (marketed as GanedenBC30®) at 1 billion colony-forming units (CFU) per 12 fl oz serving. GanedenBC30® is a spore-forming probiotic uniquely suited to carbonated beverage applications because the endospore form survives the acidic pH (approximately 2.5-3.5) and dissolved CO₂ environment of carbonated beverages throughout shelf life, germinating upon reaching the gastrointestinal tract (Endres et al., 2009; Keller et al., 2017). No energy beverage known to the inventors combines a probiotic with paraxanthine.

The inclusion of GanedenBC30® serves three mechanistically distinct functions:
Gut-brain axis modulation: The gut-brain axis—mediated by the vagus nerve, enteric nervous system, and microbial metabolite signaling—is an established bidirectional communication pathway between the gastrointestinal microbiome and the central nervous system. Bacillus coagulans produces short-chain fatty acids (SCFAs), particularly butyrate and lactate, which modulate enteric serotonergic signaling and vagal afferent activity, influencing mood, anxiety, and cognitive clarity (Dinan & Cryan, 2017; Foster & McVey Neufeld, 2013). This constitutes a fourth axis of cognitive modulation—gut-brain—distinct from the adenosinergic, cholinergic, and dopaminergic pathways addressed by the other active ingredients.
Immune support: GanedenBC30® has been clinically demonstrated to enhance immune function markers including increased T-cell activation and anti-inflammatory cytokine production (Kimmel et al., 2010), supporting overall systemic health during periods of cognitive and physical demand.
Digestive health: GanedenBC30® improves digestive comfort metrics including reduced bloating, gas, and abdominal discomfort (Hun, 2009), directly addressing a common consumer complaint with existing energy drinks.

The combination of paraxanthine-driven top-down cognitive enhancement (adenosinergic blockade in the CNS) with probiotic-mediated bottom-up gut-brain axis modulation represents a novel bidirectional cognitive optimization strategy not taught or suggested by any prior art known to the inventors. See FIG. 6.

### VII. L-tyrosine and Catecholaminergic Support

L-Tyrosine is present at 500 mg per serving as the biosynthetic precursor for dopamine, norepinephrine, and epinephrine via the tyrosine hydroxylase (TH) pathway. Under conditions of sustained cognitive demand, stress, or sleep deprivation, catecholamine synthesis becomes rate-limited by substrate availability rather than enzyme activity (Fernstrom & Fernstrom, 2007). L-Tyrosine supplementation at 500 mg has been demonstrated to maintain cognitive function under stress and multitasking conditions in military and civilian populations (Mahoney et al., 2007; Jongkees et al., 2015). In the present formulation, L-Tyrosine synergizes with paraxanthine's A2A-mediated dopaminergic disinhibition in the nucleus accumbens, providing both upstream substrate support (L-Tyrosine) and downstream release facilitation (paraxanthine), maximizing dopaminergic tone for motivation and executive function.

### VIII. Prior Art Deficiencies

The following limitations characterize the prior art known to the inventors as of the filing date:
US 20230115966 A1 (Ingenious Ingredients L.P.): Discloses paraxanthine combined with a single cholinergic agent, describing Alpha-GPC and citicoline as interchangeable alternatives. Does not disclose both simultaneously in a single serving, does not disclose taurine-paraxanthine synergy, does not disclose a probiotic component, and does not disclose a ready-to-drink carbonated beverage format.
US 20230072854 A1 (Ingenious Ingredients/PX ING): Discloses paraxanthine compositions but does not include dual cholinergic precursors, taurine at synergistic ratios, or a carbonation-stable probiotic.
WO 2023/064511 A9 (Ingenious Ingredients L.P.): Uses singular “a cholinergic agent” language without mechanistic distinction between Alpha-GPC and citicoline. Does not disclose a carbonated beverage with taurine-paraxanthine synergistic complex or probiotic integration.
US 20160038552 A1 (Buck Institute for Research on Aging, abandoned): Discloses Alpha-GPC and citicoline as discrete alternatives (claims 18-22), not simultaneously. Does not include paraxanthine or a probiotic component.
No commercial energy beverage: No commercially available energy drink known to the inventors contains paraxanthine, combines Alpha-GPC with citicoline, or includes a probiotic in a carbonated energy drink format.

See FIG. 7 for a competitive patent and product landscape gap matrix.

### SUMMARY OF THE INVENTION

In one aspect, the invention provides a ready-to-drink carbonated beverage composition comprising: (a) paraxanthine (1,7-dimethylxanthine) in a cognitively effective amount of from about 100 mg to about 500 mg per serving; (b) Alpha-glycerophosphocholine (Alpha-GPC) in a cholinergically effective amount; (c) citicoline (CDP-choline) in a cholinergically effective amount; (d) taurine in a synergistically effective amount; and (e) a carbonation-stable spore-forming probiotic; wherein Alpha-GPC and citicoline are simultaneously present in a single serving, and wherein the composition is substantially free of caffeine, theobromine, and theophylline, and is provided in a carbonated aqueous base.

The composition preferably produces cognitive and energetic benefits not achievable by any commercially available energy beverage, through the synergistic interaction of five mechanistically distinct axes: (1) adenosinergic stimulation via paraxanthine; (2) cholinergic amplitude enhancement via Alpha-GPC; (3) cholinergic structural endurance via citicoline; (4) GABAergic modulation and osmoregulatory support via taurine; and (5) gut-brain axis modulation via the spore-forming probiotic. Preferred embodiments further comprise L-tyrosine, L-theanine, Panax ginseng extract, ascorbic acid, pyridoxine, methylcobalamin, and a zero-sugar sweetener system. Methods of cognitive enhancement, sustained mental energy delivery, and gut-brain axis modulation are also disclosed.

### BRIEF DESCRIPTION OF THE DRAWINGS

FIG. 1 is a pie chart showing caffeine metabolic yield distribution (CYP1A2-mediated): paraxanthine~84%, theobromine~12%, theophylline~4%.

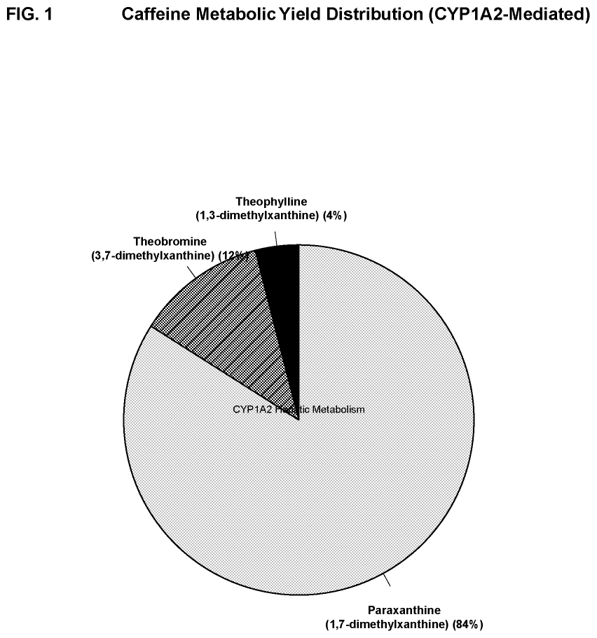

FIG. 2 is a schematic line graph, illustrative and not based on direct clinical measurement, showing comparative pharmacokinetic profiles of caffeine 300 mg oral versus paraxanthine 300 mg oral in a beverage format, constructed from known pharmacokinetic parameters reported in the literature to depict the relative time-to-peak, peak concentration, and elimination phase differences.

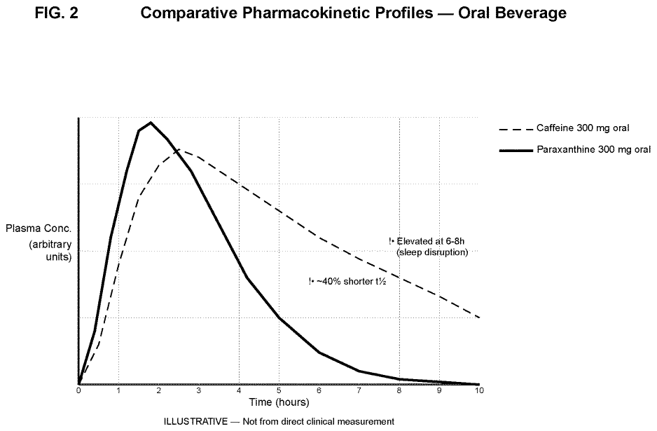

FIG. 3 is a bar chart showing active ingredient doses per 12 fl oz (355 mL) serving for the nine milligram-scale active components grouped by neurochemical system, with Vitamin B12 (6 mcg) and GanedenBC30® (1 Billion CFU) noted separately due to different measurement unit scales.

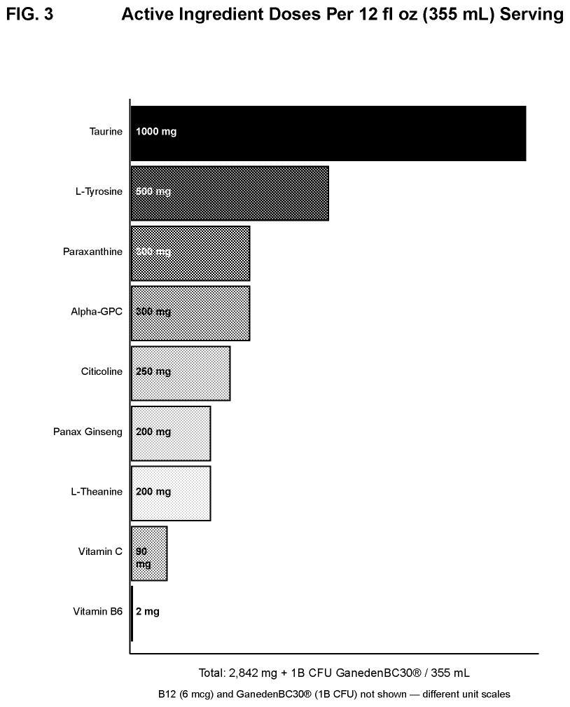

FIG. 4 is a flowchart illustrating the Dual Choline Architecture and Tripartite Cholinergic Enhancement Mechanism as applied to the beverage format. Reference numerals designate: [100] ARM 1 (Rapid Amplitude, Alpha-GPC arm); [102] Paraxanthine (1,7-dimethylxanthine); [104] ARM 2 (Structural Endurance, Citicoline arm); [106] Alpha-GPC 300 mg; [108] blood-brain barrier crossing via lysolecithin transporter; [110] intraneuronal Phospholipase D hydrolysis; [112] ChAT-mediated acetylcholine synthesis; [114] peak ACh amplitude output (Vector 2); [116] Citicoline (CDP-choline) 250 mg; [118] systemic hydrolysis to choline and cytidine; [120] cytidine-to-uridine conversion and CNT2 transport; [122] Kennedy pathway phosphatidylcholine biosynthesis; [124] membrane structural capacity output (Vector 3); [126] Adenosine A1 receptor antagonism on basal forebrain cholinergic neurons; [128] presynaptic acetylcholine release disinhibition (Vector 1); [132] Tripartite Cholinergic Output node; [134] Cognitive Outcome.

FIG. 5 is a schematic diagram illustrating the Taurine-Paraxanthine Synergistic Complex and its four mechanistic arms: GABAergic modulation, calcium homeostasis, osmoregulation, and anti-inflammatory neuroprotection, converging on jitter-free sustained alertness.

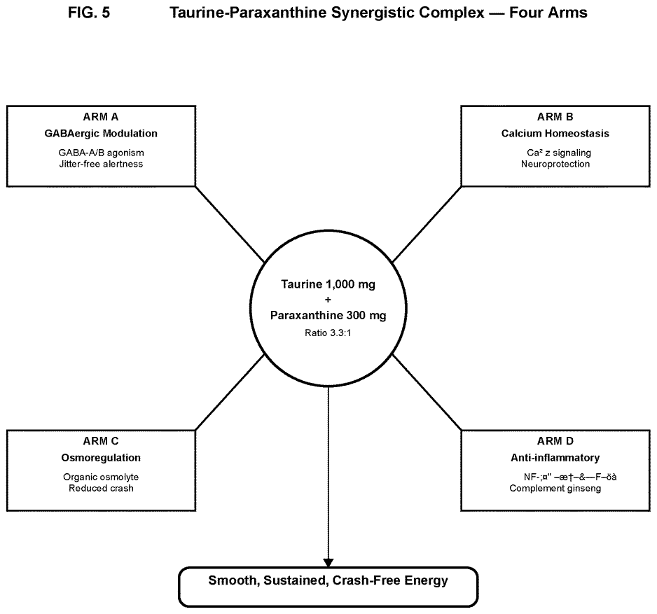

FIG. 6 is a schematic diagram illustrating the Bidirectional Cognitive Optimization Strategy: top-down CNS stimulation via paraxanthine (adenosinergic blockade) and bottom-up gut-brain axis modulation via GanedenBC30® probiotic (SCFA production, vagal afferent signaling, enteric serotonergic modulation).

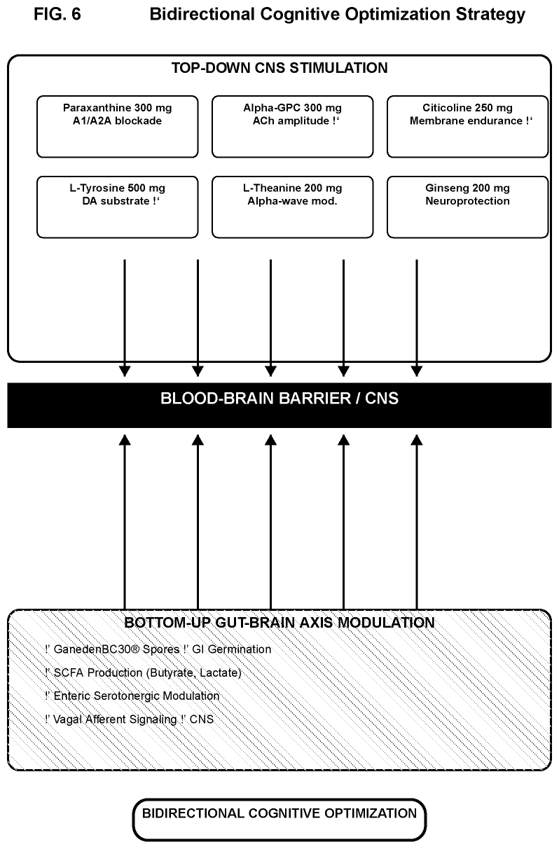

FIG. 7 is a competitive patent and product landscape gap matrix comparing the present invention to prior art US20230115966, US20230072854, WO2023/064511, US20160038552A1, and commercial energy beverages (Red Bull, Monster, Celsius, C4).

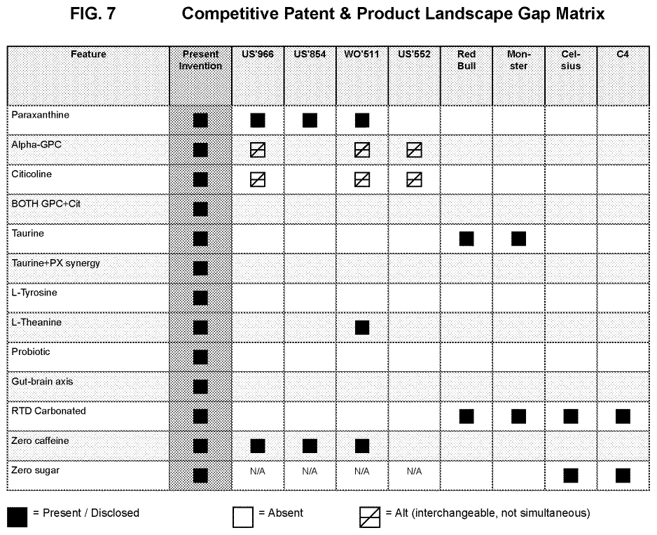

FIG. 8 is a radar chart illustrating neurochemical system coverage on eight axes for the preferred eleven-compound embodiment: Adenosinergic, Cholinergic-Amplitude, Cholinergic-Structural, Dopaminergic, GABAergic, Neuroprotective, Gut-Brain, and Osmoregulatory.

FIG. 9 is a schematic cross-sectional view of the beverage can format illustrating the carbonated aqueous matrix, dissolved active ingredient distribution, CO₂ saturation zone, and GanedenBC30® endospore dispersion pattern within the carbonated environment.

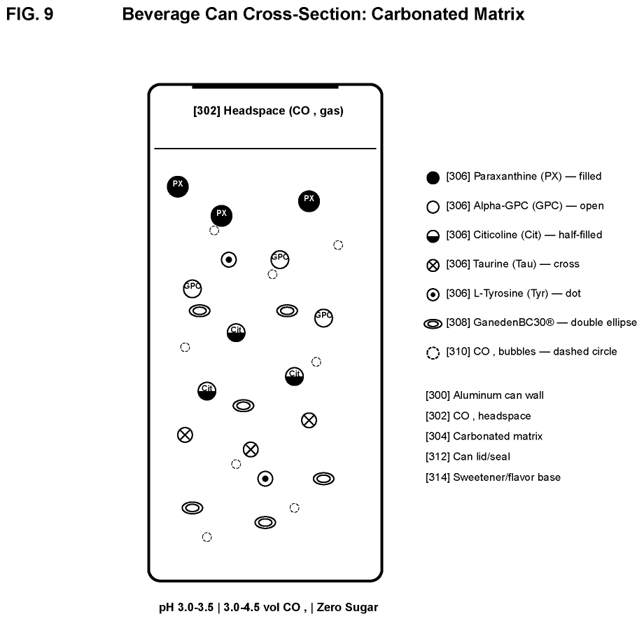

FIG. 10 is a bar chart comparing GanedenBC30® endospore survival rates in carbonated acidic beverage matrix (pH 3.0-3.5) versus vegetative-cell probiotics (Lactobacillus spp.) over a 12-month shelf life, demonstrating >90% spore viability in the carbonated format.

FIG. 11 is a schematic diagram illustrating the L-Tyrosine catecholaminergic pathway showing tyrosine hydroxylase (TH)-mediated conversion to L-DOPA, AADC-mediated conversion to dopamine (with PLP cofactor from pyridoxine), and downstream norepinephrine synthesis, with paraxanthine A2A-mediated dopaminergic disinhibition at the nucleus accumbens.

### DETAILED DESCRIPTION OF THE PREFERRED EMBODIMENTS

### Section 1—Definitions and Amounts

As used throughout this specification and in the claims:
“Cognitively effective amount” of paraxanthine: from about 100 mg to about 500 mg per serving, preferred about 300 mg.
“Cholinergically effective amount” of Alpha-GPC: from about 100 mg to about 600 mg per serving, preferred about 300 mg.
“Cholinergically effective amount” of citicoline: from about 100 mg to about 500 mg per serving, preferred about 250 mg.
“Synergistically effective amount” of taurine: from about 500 mg to about 2,000 mg per serving, preferred about 1,000 mg, at a taurine-to-paraxanthine weight ratio of from about 2:1 to about 5:1, preferred about 3.3:1.
“Dual Choline Architecture”: the simultaneous co-administration of both Alpha-GPC and citicoline in a single serving, wherein Alpha-GPC acts through phospholipase D for ChAT-mediated ACh synthesis (amplitude arm) and citicoline acts through the Kennedy pathway for phosphatidylcholine membrane biosynthesis (structural endurance arm), the two precursors being mechanistically non-redundant.
“Mechanistically non-redundant”: acting through different molecular substrates, enzymatic pathways, temporal scales, and physiological failure modes such that doubling either compound alone cannot replicate the combined effect of both simultaneously administered.
“Tripartite Cholinergic Enhancement”: three-vector simultaneous cholinergic modulation achieved by paraxanthine (A1 receptor block disinhibiting ACh release), Alpha-GPC (elevating ACh synthesis amplitude), and citicoline (maintaining membrane phosphatidylcholine via Kennedy pathway), each vector being mechanistically distinct.
“Synergistic” or “synergy” as used herein means that the combined effect of two or more compounds on a specified biological endpoint exceeds the sum of the individual effects of each compound administered separately at the same dose; i.e., the combination produces a supra-additive response. Where direct clinical measurement of a synergistic effect is not available, the term is used based on mechanistic non-redundancy: the compounds act through distinct molecular targets, enzymatic pathways, or receptor systems such that the combination addresses biological endpoints inaccessible to any single compound regardless of dose.
“Taurine-Paraxanthine Synergistic Complex”: the combination of taurine and paraxanthine at a weight ratio of approximately 2:1 to approximately 5:1, producing balanced GABAergic-adenosinergic modulation characterized by sustained alertness without jitter, anxiety, or post-peak crash.
“Carbonation-stable spore-forming probiotic”: a probiotic organism in endospore form that maintains viability (>90% CFU retention) in a carbonated aqueous environment at pH 2.5-4.0 and dissolved CO₂ concentration of 3.0-5.0 volumes over a shelf life of at least 6 months at ambient temperature.
“Gut-brain axis modulation”: modulation of central nervous system function through gastrointestinal microbiome-mediated pathways including short-chain fatty acid (SCFA) production, vagal afferent signaling, and enteric serotonergic modulation.
“Substantially free of caffeine, theobromine, and theophylline”: less than 5 mg total of the three compounds combined per serving.
“Serving”: the total content of a single unit container (12 fl oz/355 mL can), intended for single consumption.
“Ready-to-drink carbonated beverage”: an aqueous composition dissolved in carbonated filtered water, packaged in a sealed container, and requiring no further preparation prior to oral consumption.
“Bidirectional Cognitive Optimization”: simultaneous top-down CNS stimulation via paraxanthine and related compounds acting centrally, and bottom-up gut-brain axis modulation via probiotic-mediated enteric signaling, producing a cognitive benefit not achievable by either approach alone.
GanedenBC30® is a registered trademark of Kerry Group plc (formerly Ganeden, Inc.). Use of the trademark herein is for identification purposes only and does not imply endorsement by or affiliation with the trademark owner. The generic designation Bacillus coagulans GBI-30, 6086 is used interchangeably throughout this specification and in the claims.

### Section 2—Mechanistic Non-Redundancy of Alpha-GPC and Citicoline (Table 1)

Table 1 demonstrates that Alpha-GPC and citicoline are non-interchangeable at any dose, each addressing distinct failure modes of cholinergic neurotransmission. The mechanistic analysis set forth in Table 1 of the co-pending application No. Ser. No. 19/630,373 is incorporated herein by reference and applies equally to the oral beverage doses of the present invention.

Molecular substrate: Alpha-GPC=L-alpha-glycerylphosphorylcholine; Citicoline=CDP-choline (cytidine-5′-diphosphocholine). Rate-limiting enzyme: Alpha-GPC=Phospholipase D (intraneuronal); Citicoline=CTP: phosphocholine cytidylyltransferase. Active metabolite: Alpha-GPC=Free choline; Citicoline=Cytidine converted to Uridine (plasma). CNS delivery: Alpha-GPC=Lysolecithin transporter (intact molecule); Citicoline=CNT2 nucleoside transporter (uridine). Target pathway: Alpha-GPC=ChAT: choline+acetyl-CoA→ACh; Citicoline=Kennedy: CTP→CDP-choline→PC. Physiological effect: Alpha-GPC=Peak ACh synthesis amplitude increased; Citicoline=Neuronal membrane PC pool maintained. Failure mode corrected: Alpha-GPC=Insufficient peak ACh per synaptic event; Citicoline=Membrane degradation under sustained firing. Temporal scale: Alpha-GPC=Rapid: 30-60 minutes; Citicoline=Sustained: hours (membrane replenishment). Replicated by 2× other alone?=No for both.

### Section 3—Paraxanthine as the Third Cholinergic Vector

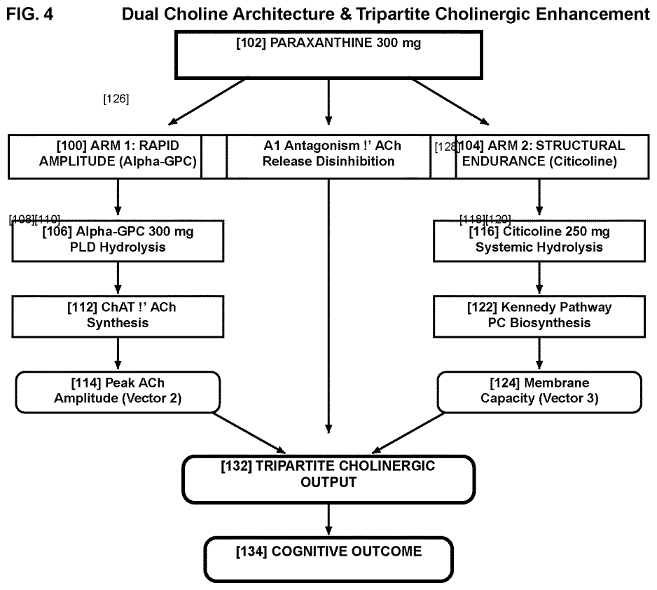

Adenosine A1 receptors are expressed presynaptically on basal forebrain cholinergic neurons of the Ch1-Ch4 cell groups projecting to the hippocampus and prefrontal cortex. Tonic adenosine A1 activation at physiological resting adenosine concentrations of approximately 100-200 nM tonically suppresses basal forebrain ACh release. Paraxanthine's competitive antagonism of A1 receptors directly disinhibits this suppression, increasing presynaptic ACh release independent of ACh synthesis capacity or membrane substrate availability (Daly et al., 1993; Fredholm et al., 1999; Moran et al., 2019). This constitutes a third mechanistically distinct cholinergic vector—ACh release disinhibition—not provided by either Alpha-GPC or citicoline, and not further enhanced by doubling either precursor dose. At 300 mg oral dose, paraxanthine achieves robust A1 and A2A antagonism supporting both the Tripartite Cholinergic Enhancement and the dopaminergic facilitation described in Section 7. See FIG. 4.

### Section 4—Preferred Embodiment (Table 2)

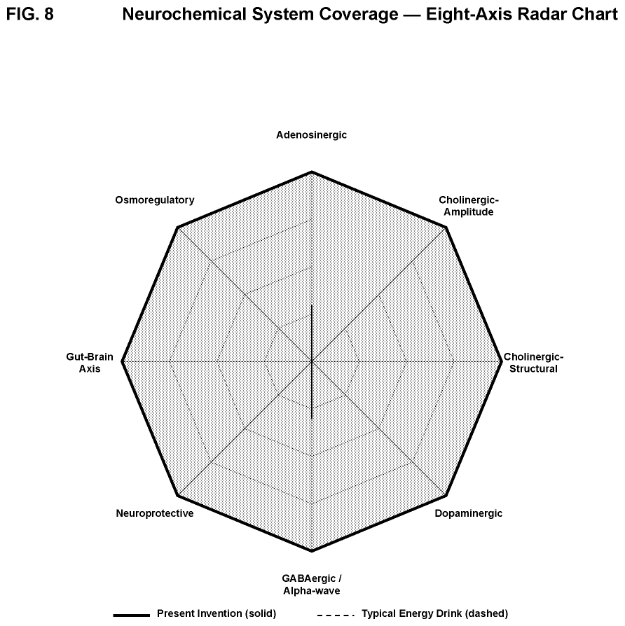

The preferred embodiment is a ready-to-drink carbonated beverage comprising the composition set forth in Table 2, providing full-spectrum cognitive enhancement across eight neurochemical and physiological systems simultaneously. See FIG. 8.

Table 2—Preferred Composition Per 12 fl oz (355 mL) Serving:
Taurine—1,000 mg—GABA-A/B agonism; calcium homeostasis; osmoregulation; anti-inflammatory—GABAergic; Osmoregulatory
Paraxanthine (1,7-dimethylxanthine)—300 mg—Adenosine A1/A2A antagonist; ACh release disinhibition; dopaminergic/noradrenergic—Adenosinergic; Dopaminergic; Cholinergic-Release
L-Tyrosine—500 mg—Tyrosine hydroxylase substrate; dopamine and norepinephrine precursor—Dopaminergic; Catecholaminergic
Alpha-GPC—300 mg—Phospholipase D→free choline→ChAT-mediated ACh synthesis (amplitude arm)—Cholinergic-Amplitude
Citicoline (CDP-choline)—250 mg—Cytidine→uridine→Kennedy pathway→phosphatidylcholine biosynthesis (structural arm)—Cholinergic-Structural
Panax Ginseng Extract—200 mg—Ginsenoside Rg1/Rb1: hippocampal arborization; NF-κB inhibition; mitochondrial ROS reduction—Neuroprotective
L-Theanine—200 mg—Alpha-wave (8-13 Hz) promotion; attenuates stimulant-associated hyperarousal; 2:3 ratio with paraxanthine—GABAergic/Alpha-wave
GanedenBC30® (Bacillus coagulans GBI-30, 6086)—1 Billion CFU—Spore-stable in carbonation; SCFA production; vagal afferent modulation; gut-brain axis—Gut-Brain Axis
Vitamin C (Ascorbic Acid)—90 mg (100% DV)—Antioxidant; cofactor for dopamine beta-hydroxylase (norepinephrine synthesis)—Antioxidant; Catecholaminergic cofactor
Vitamin B6 (Pyridoxine)—2 mg (118% DV)—PLP cofactor for AADC (catecholamine/serotonin biosynthesis under elevated demand)—Catecholaminergic cofactor
Vitamin B12 (Methylcobalamin)—6 mcg (250% DV)—SAMe synthesis; Kennedy pathway CTP: phosphocholine cytidylyltransferase support—Methylation cofactor

Total active ingredient load: 2,842 mg+1 Billion CFU GanedenBC30® probiotic per 12 fl oz (355 mL) serving, representing less than 1% by weight of the total beverage volume and leaving substantial formulation headroom for the carbonated water base, flavoring, sweetener system, and preservative components.

Beverage base: Carbonated Filtered Water, Citric Acid, Natural Flavors, Steviol Glycosides, Monk Fruit Extract, Sea Salt (35 mg sodium), Potassium Sorbate. Zero sugar, zero caffeine, fully disclosed label.

### Section 5—Unexpected Results and Non-obviousness

The following unexpected results support the non-obviousness of the present invention under 35 U.S.C. § 103:
The simultaneous presence of both Alpha-GPC (300 mg) and citicoline (250 mg) at oral doses in a carbonated beverage addresses two neurochemical failure modes that cannot be corrected by either precursor alone at any dose, as established in Table 1. This produces a sustained high-performance cognitive state with reduced post-peak deficit not observed with either precursor alone.
The taurine-paraxanthine synergistic complex at a 3.3:1 weight ratio produces a jitter-free, crash-free energy profile not achievable by paraxanthine alone or by taurine combined with caffeine. The GABAergic modulation provided by taurine specifically counterbalances paraxanthine's adenosinergic excitation without diminishing alertness—a result that would not have been predicted from the prior art teaching taurine-caffeine combinations.
The incorporation of GanedenBC30® as a carbonation-stable probiotic creates a bidirectional cognitive optimization strategy (top-down CNS stimulation+bottom-up gut-brain axis modulation) never previously combined in an energy beverage. The gut-brain axis contribution to cognitive clarity represents a fourth axis of cognitive modulation orthogonal to the adenosinergic, cholinergic, and dopaminergic pathways.
The L-Tyrosine-paraxanthine combination provides both upstream substrate support and downstream release facilitation for dopaminergic tone—a dual-vector dopaminergic strategy not taught by any energy drink formulation in the prior art.
The L-Theanine to paraxanthine ratio of 2:3 (200 mg: 300 mg) is specifically optimized for paraxanthine's shorter half-life and cleaner pharmacokinetic profile, as distinct from the well-known 2:1 L-theanine-to-caffeine ratio used in tea-derived studies. This ratio produces a ‘cleaner landing’—reduced rebound anxiety and smoother cognitive offset—specific to paraxanthine's metabolic profile.
No prior art reference, combination of references, or clinical study teaches, suggests, or discloses the simultaneous administration of paraxanthine, Alpha-GPC, citicoline, taurine, L-tyrosine, and a carbonation-stable probiotic in a single ready-to-drink carbonated beverage.

### Section 6—Adjunct Ingredient Scientific Basis

L-Theanine (preferred 200 mg; paraxanthine: theanine weight ratio 3:2): Promotes alpha-band (8-13 Hz) cortical oscillations, attenuates adenosine-blockade-induced cortical hyperarousal, and reduces anxiety-like subjective responses to stimulant compounds without impairing vigilance (Haskell et al., 2008; Nobre et al., 2008). The 3:2 ratio with paraxanthine (as opposed to the conventional 1:2 caffeine: theanine ratio) reflects paraxanthine's shorter half-life and reduced peripheral side-effect profile, requiring less theanine buffering than caffeine.
Panax Ginseng Root Extract (preferred≥20% ginsenosides; 200 mg): Ginsenoside Rg1 promotes hippocampal dendritic arborization and long-term potentiation; Rb1 reduces mitochondrial reactive oxygen species; ginsenosides broadly inhibit NF-κB and downstream neuroinflammatory cytokines (Kennedy et al., 2001). The 200 mg dose in the beverage format provides robust neuroprotective coverage during the paraxanthine activity window.
Ascorbic Acid (Vitamin C) (preferred 90 mg, 100% DV): Beyond its well-known antioxidant function, ascorbic acid is a required cofactor for dopamine beta-hydroxylase (DBH), the enzyme catalyzing the conversion of dopamine to norepinephrine. Under L-Tyrosine-driven elevated dopamine synthesis, ascorbic acid ensures that the dopamine-to-norepinephrine conversion step is not rate-limited by cofactor availability.
Pyridoxine (Vitamin B6) (preferred 2 mg, 118% DV based on current FDA reference daily value of 1.7 mg): Under paraxanthine-driven elevation of catecholaminergic firing, aromatic L-amino acid decarboxylase (AADC)—pyridoxal-5′-phosphate (PLP)-dependent—becomes rate-limiting for dopamine and serotonin synthesis. Supplemental pyridoxine ensures PLP availability does not become rate-limiting under elevated demand.
Methylcobalamin (Vitamin B12) (preferred 6 mcg, 250% DV): Serves as cofactor for methionine synthase in S-adenosylmethionine (SAMe) synthesis, supporting COMT-mediated catecholamine catabolism and Kennedy pathway methylation step, directly supporting Arm 2 of the Dual Choline Architecture under high-turnover conditions.
Sea Salt (preferred 35 mg sodium): Provides trace electrolyte support for neural signal conduction and cellular hydration, complementing taurine's osmoregulatory function.

### Section 7—L-tyrosine and Dual-vector Dopaminergic Enhancement

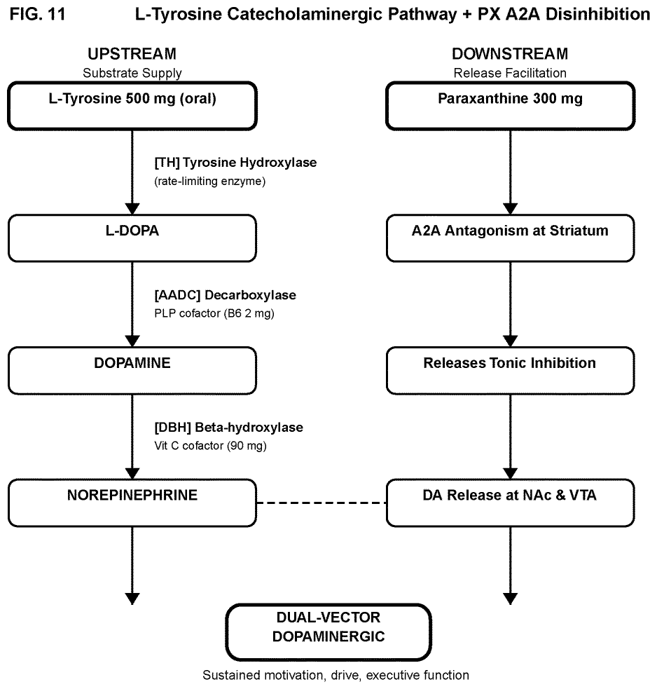

The present invention discloses a dual-vector dopaminergic enhancement strategy uniquely enabled by the combination of L-Tyrosine (500 mg) and paraxanthine (300 mg):
Vector 1—Upstream Substrate Supply (L-Tyrosine): L-Tyrosine is hydroxylated by tyrosine hydroxylase (TH) to L-DOPA, which is decarboxylated by AADC (PLP-dependent) to dopamine. Under baseline conditions, TH activity is the rate-limiting step; however, under sustained cognitive demand or stress, substrate depletion can become rate-limiting. 500 mg L-Tyrosine ensures adequate substrate availability for sustained dopamine synthesis. See FIG. 11.
Vector 2—Downstream Release Facilitation (Paraxanthine): Paraxanthine's competitive antagonism of A2A receptors in the striatum releases tonic adenosinergic inhibition of dopaminergic neurons in the nucleus accumbens and ventral tegmental area (VTA), increasing dopamine release and postsynaptic D1/D2 receptor activation. This A2A-mediated disinhibition is mechanistically distinct from substrate supply and cannot be replicated by increasing L-Tyrosine dose alone.

The dual-vector approach produces sustained motivational drive and executive function enhancement not achievable by either compound alone, as the upstream supply ensures that the downstream facilitation has adequate substrate to act upon.

### Section 8—Carbonated Beverage Formulation and Manufacturing

The composition of the present invention is formulated as a ready-to-drink carbonated beverage in a 12 fluid ounce (355 mL) aluminum can format. The non-limiting exemplary method of preparation is set forth below.
Step 1—Active Ingredient Premix Preparation. Each water-soluble active ingredient is weighed individually to the target dose using a calibrated analytical balance (±0.5 mg accuracy or better): Taurine 1,000 mg; Paraxanthine 300 mg; L-Tyrosine 500 mg; Alpha-GPC 300 mg; Citicoline 250 mg; Panax Ginseng Extract (≥20% ginsenosides) 200 mg; L-Theanine 200 mg; Ascorbic Acid 90 mg; Pyridoxine 2 mg; Methylcobalamin 6 mcg. Identity and purity of each ingredient are confirmed against certificate of analysis (CoA) from the supplier prior to use. Paraxanthine is confirmed by HPLC-UV (λ=274 nm) against a validated reference standard. Alpha-GPC is confirmed by enzymatic choline assay (≥97% assay). The active ingredient premix is dissolved in a portion of the filtered water base under controlled agitation (100-200 rpm, impeller mixer) at 20-25° C. until fully dissolved. Alpha-GPC, being hygroscopic, is added last to the premix to minimize clumping.
Step 2—Probiotic Addition. GanedenBC30® (Bacillus coagulans GBI-30, 6086) endospores are added at a target concentration of 1 Billion CFU per 355 mL serving. Endospores are added to the premix solution at ≤25° C. to prevent premature germination. The spore-forming nature of GanedenBC30® ensures viability in the acidic (pH 3.0-3.5) carbonated environment throughout the target shelf life of 12-18 months. Viability is confirmed by plate count assay (TSA agar, 37° C., 48 h) at time points of 0, 3, 6, 9, and 12 months.
Step 3—Sweetener and Flavor System. Steviol glycosides (stevia extract, ≥95% Reb A) and monk fruit extract (mogrosides V, ≥50%) are added in combination as a zero-sugar, zero-calorie sweetener system. Natural flavoring agents are added according to the selected SKU: Mango Peach, Strawberry Kiwi, Blue Raspberry, or Watermelon. Citric acid is added to achieve target pH of 3.0-3.5. Sea salt (35 mg sodium equivalent) is dissolved in the base. Potassium sorbate is added at ≤1,000 ppm as a preservative.
Step 4—Carbonation. The complete beverage solution is carbonated to 3.0-4.5 volumes CO₂ using a counter-pressure carbonation system at 2-4° C. Carbonation is performed after all active ingredients and probiotic spores are dissolved/suspended to ensure uniform distribution. The low carbonation temperature minimizes paraxanthine degradation and maintains GanedenBC30® spore dormancy.
Step 5—Filling and Sealing. The carbonated beverage is filled into 12 fl oz (355 mL) aluminum cans using a counter-pressure filler to maintain carbonation levels. Cans are sealed with standard 202-diameter ends. Fill volume tolerance: ±2% of nominal 355 mL. In-process checks include fill level verification (gamma or X-ray), seam integrity (double seam teardown), and dissolved CO₂ measurement every 30 minutes during production.
Step 6—Quality Control Testing. Finished cans are sampled per a validated sampling plan and subjected to: (a) active ingredient content verification by HPLC (paraxanthine, Alpha-GPC, citicoline, L-Tyrosine, taurine, L-Theanine); acceptance criteria: each analyte 90.0-110.0% of label claim; (b) GanedenBC30® viability by plate count (≥1 Billion CFU per serving at time of manufacture, with ≥90% retention at 12 months ambient storage); (c) pH verification (3.0-3.5); (d) dissolved CO₂ (3.0-4.5 volumes); (e) microbial enumeration (total aerobic plate count ≤100 CFU/mL; yeast and mold ≤10 CFU/mL; absence of Salmonella, E. coli, Staphylococcus aureus); (f) heavy metals—arsenic ≤1.5 ppm, cadmium ≤0.5 ppm, lead ≤0.5 ppm, mercury ≤0.1 ppm per USP <232>.
Step 7—Packaging and Labeling. Cans are packaged in 4-pack, 8-pack, 12-pack, and 24-pack secondary packaging with labeling compliant with applicable FDA dietary supplement and/or conventional food labeling requirements (21 CFR Part 101), including a Supplement Facts panel or Nutrition Facts panel as applicable, ingredient list, allergen declaration, net quantity, and lot number/expiration date. Storage conditions: ambient temperature (15-25° C.), away from direct sunlight. Stability studies conducted under ICH Q1A(R2) accelerated conditions (40° C./75% RH, 6 months) and long-term conditions (25° C./60% RH, 18 months) are used to establish expiration dating.

### Section 9—Flavor Embodiments

The preferred embodiment is available in four alternative flavor embodiments, each comprising the same active ingredient composition set forth in Table 2 with a distinct natural flavor system:
SKU 1—Mango Peach: Tropical sweetness with soft stone fruit character. Broad consumer appeal. Designated hero/launch flavor.
SKU 2—Strawberry Kiwi: Sweet strawberry with tart exotic kiwi. Premium nostalgic profile with strong female and Gen Z demographic appeal.
SKU 3—Blue Raspberry: Bold, punchy, candy-forward flavor profile. Optimized for social media shareability and gym/gaming/student demographics.
SKU 4—Watermelon: Clean, refreshing, crushable. Year-round versatility. The flavor system in each SKU is formulated to mask the mild bitterness of paraxanthine and the slight metallic note of Alpha-GPC without impairing the dissolution or bioavailability of any active ingredient.

### Section 10—Methods of Administration and Expected Cognitive Timeline

The beverage is consumed orally. A single serving (one 12 fl oz can) is consumed in one session, typically within 10-30 minutes. The full active ingredient load is absorbed via normal gastrointestinal transit and hepatic processing.

Expected cognitive timeline based on known pharmacokinetic parameters of the individual active ingredients:
0-15 minutes: Taurine and L-Theanine absorption initiates; GABAergic tone begins modulating baseline anxiety/arousal state.
15-45 minutes: Paraxanthine reaches peak plasma concentration (Tmax~45 min oral); adenosinergic blockade and dopaminergic disinhibition engage. Alpha-GPC begins releasing free choline for ACh synthesis.
45-90 minutes: Full tripartite cholinergic enhancement active. L-Tyrosine-driven dopamine synthesis at peak. Citicoline-derived uridine entering Kennedy pathway for membrane biosynthesis. Peak cognitive performance window.
90-180 minutes: Sustained performance plateau. Citicoline structural endurance arm maintains membrane capacity. Taurine osmoregulation supports continued neuronal firing.
180-240 minutes: Gradual offset. Paraxanthine half-life (~3.5h) produces smooth decline without crash. L-Theanine provides ‘clean landing’ via alpha-wave promotion.
Ongoing: GanedenBC30® probiotic spores germinate in the GI tract, contributing to gut-brain axis modulation over hours to days with cumulative daily use.

Primary adenosinergic effect duration is approximately 3-4 hours, corresponding to paraxanthine's half-life of approximately 3.5 hours. The shorter half-life relative to caffeine (~5-7 hours) materially reduces sleep disruption risk for afternoon consumption.

### Section 11—Dose Rationale for Oral Beverage Format

The active ingredient doses in the present beverage formulation are specifically calibrated for oral bioavailability. Published clinical trials demonstrating cognitive effects of Alpha-GPC have employed oral doses of 300-630 mg per administration. The 300 mg Alpha-GPC dose in the present formulation falls within the clinically validated oral range. Citicoline at 250 mg orally provides adequate substrate for Kennedy pathway activation. Paraxanthine at 300 mg orally provides robust adenosinergic blockade accounting for first-pass metabolism. These oral doses are materially higher than the transmucosal doses in the co-pending pouch application (Alpha-GPC 60 mg, citicoline 60 mg, paraxanthine 120 mg), reflecting the route-dependent bioavailability differential between transmucosal and gastrointestinal absorption.

The taurine dose (1,000 mg) is consistent with existing energy drink dosing (Red Bull 1,000 mg, Monster 1,000 mg) but is here combined for the first time with paraxanthine rather than caffeine, producing a distinct synergistic profile as described in Background Section V.

The L-Tyrosine dose (500 mg) is within the range demonstrated effective for cognitive maintenance under stress in peer-reviewed clinical trials (150-2,000 mg; Mahoney et al., 2007).

### Section 12—Stability and Shelf Life

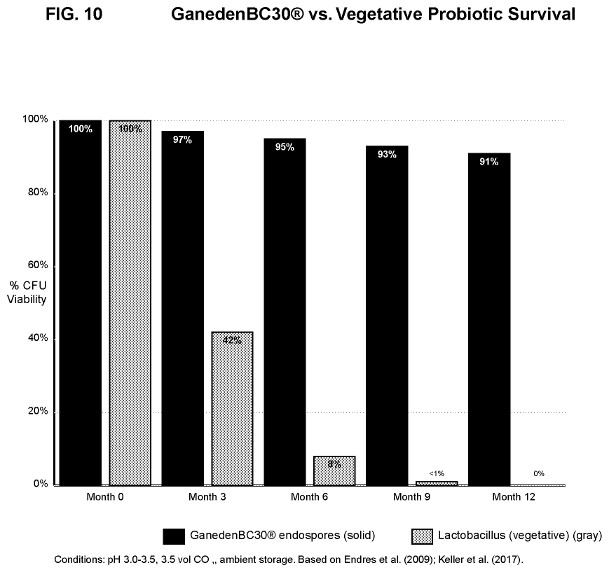

Paraxanthine is chemically stable in aqueous acidic conditions (pH 3.0-3.5) at ambient temperature. Alpha-GPC is hygroscopic in powder form but stable once dissolved in aqueous solution at acidic pH. Citicoline demonstrates good aqueous stability at pH 3.0-4.0. GanedenBC30® endospores are specifically selected for carbonated beverage applications due to their demonstrated stability in acidic, carbonated matrices; published shelf-life studies show >90% spore viability at 12 months ambient storage in carbonated beverages (Endres et al., 2009). Keller et al. (2017) further demonstrated the functional activity of Bacillus coagulans GBI-30, 6086 in acidic aqueous environments, confirming endospore resilience under conditions consistent with carbonated beverage matrices. See FIG. 10. The aluminum can format provides complete light barrier and oxygen barrier properties, minimizing photodegradation and oxidative degradation of ascorbic acid and other light-sensitive components.

### Section 13—Prophetic Comparative Example

The following prophetic example is provided to further illustrate the expected superior performance of the present invention relative to partial formulations. This example has not been reduced to practice but is based on the established pharmacological profiles and published clinical data for each active ingredient as described in the preceding sections.

### Prophetic Example 1—Comparative Cognitive Assessment

Four groups (n=20 per group) of healthy adult subjects (ages 21-45, BMI 18.5-30, non-smokers, caffeine-naive for 48 hours prior) are administered, in a randomized double-blind crossover design with 7-day washout periods, the following formulations in 12 fl oz carbonated beverages:
Group A (PX Only): Paraxanthine 300 mg in carbonated base. No cholinergic, GABAergic, dopaminergic, or probiotic components.
Group B (PX+Single Choline): Paraxanthine 300 mg+Alpha-GPC 300 mg in carbonated base. Single cholinergic arm only.
Group C (PX+Dual Choline): Paraxanthine 300 mg+Alpha-GPC 300 mg+Citicoline 250 mg in carbonated base. Dual cholinergic arms, no taurine or probiotic.
Group D (Full Formulation per Table 2): All eleven active ingredients at preferred doses as set forth in Table 2, including Taurine 1,000 mg, L-Tyrosine 500 mg, L-Theanine 200 mg, Panax Ginseng 200 mg, GanedenBC30® 1B CFU, Vitamin C 90 mg, Vitamin B6 2 mg, Vitamin B12 6 mcg.

Primary endpoints: (1) Stroop test performance (reaction time, accuracy) at 30, 60, 120, and 240 minutes post-ingestion; (2) N-back working memory task (2-back, 3-back) at the same time points; (3) subjective alertness, jitter, and crash scores via Visual Analog Scale (VAS) at 30-minute intervals for 6 hours; (4) Pittsburgh Sleep Quality Index (PSQI) for the night following administration.

### Predicted Outcomes Based on the Mechanistic Analysis Set Forth in This Specification:

Group A is expected to show significant alertness improvement at 30-60 minutes (paraxanthine adenosinergic blockade) but declining performance by 180 minutes and reports of residual jitter due to uncompensated excitatory drive.
Group B is expected to show improved working memory at 60-120 minutes relative to Group A (Alpha-GPC cholinergic amplitude support) but declining sustained performance by 180-240 minutes due to membrane phospholipid depletion under sustained high-frequency cholinergic firing not corrected by Alpha-GPC alone.
Group C is expected to show sustained working memory performance through 240 minutes (citicoline Kennedy pathway membrane replenishment), but continued reports of jitter and elevated subjective anxiety scores relative to Group D due to absence of taurine GABAergic counterbalance.
Group D (full formulation) is expected to show: (i) the highest sustained Stroop and N-back performance across all time points through 240 minutes; (ii) the lowest jitter and anxiety VAS scores at all time points; (iii) the smoothest subjective offset (lowest crash scores at 4-6 hours); (iv) no significant PSQI impairment (paraxanthine's shorter half-life); and (v) cumulative gut-brain axis benefits on mood and cognitive clarity with repeated daily administration over 7+ days.

The predicted superiority of Group D over Groups A, B, and C is not merely additive but synergistic, as the five mechanistically distinct axes interact to produce a cognitive state qualitatively different from partial formulations: sustained high-amplitude cholinergic firing with intact membranes, smooth alertness without jitter, robust dopaminergic drive with adequate substrate, and emerging gut-brain axis contributions. This result would not have been obvious to a person of ordinary skill in the art from any single reference or combination of references in the prior art.

### Section 14—Terminal Disclaimer Readiness

The inventors acknowledge that the present application shares the Dual Choline Architecture concept with co-pending U.S. application Ser. No. 19/630,373 (transmucosal oral pouch) and U.S. application Ser. No. 19/636,926 (oral tablet for headache relief). While the present claims are directed to a materially different dosage form (ready-to-drink carbonated beverage), different dose ranges (oral bioavailability-adjusted doses 2.5-5× higher than transmucosal), different additional active ingredients (taurine, L-tyrosine, GanedenBC30® probiotic), and different methods of use (cognitive enhancement via beverage consumption with gut-brain axis modulation), the applicants are prepared to file a terminal disclaimer under 37 C.F.R. § 1.321(c) if a non-statutory double patenting rejection is issued, tying the term of any patent issuing from this application to the term of any patent issuing from the co-pending applications.

### Scope and Equivalents

The foregoing description of preferred embodiments has been provided for purposes of illustration and description. It is not intended to be exhaustive or to limit the invention to the precise forms disclosed. Obvious modifications and variations are possible in light of the above teachings. The embodiments were chosen and described in order to best explain the principles of the invention and its practical application, thereby enabling others skilled in the art to best utilize the invention in various embodiments and with various modifications as are suited to the particular use contemplated. It is intended that the scope of the invention be defined by the claims appended hereto and their equivalents.

What is claimed is:

1. A ready-to-drink carbonated beverage composition for cognitive enhancement comprising:
(a) paraxanthine (1,7-dimethylxanthine) in a cognitively effective amount of from about 100 mg to about 500 mg per serving;
(b) Alpha-glycerophosphocholine (Alpha-GPC) in a cholinergically effective amount of from about 100 mg to about 600 mg per serving;
(c) citicoline (CDP-choline) in a cholinergically effective amount of from about 100 mg to about 500 mg per serving;
(d) taurine in a synergistically effective amount of from about 500 mg to about 2,000 mg per serving;
(e) a carbonation-stable spore-forming probiotic at a concentration of at least 500 million CFU per serving;
wherein Alpha-GPC and citicoline are simultaneously present in the same single serving, and wherein the composition is dissolved in a carbonated aqueous base and is substantially free of caffeine, theobromine, and theophylline.

2. The composition of claim 1, wherein paraxanthine is at about 300 mg, Alpha-GPC is at about 300 mg, citicoline is at about 250 mg, and taurine is at about 1,000 mg per serving.

3. The composition of claim 1, wherein the taurine-to-paraxanthine weight ratio is from about 2:1 to about 5:1.

4. The composition of claim 3, wherein the taurine-to-paraxanthine weight ratio is about 3.3:1.

5. The composition of claim 1, further comprising L-tyrosine in an amount of from about 200 mg to about 1,000 mg per serving.

6. The composition of claim 5, wherein L-tyrosine is at about 500 mg per serving.

7. The composition of claim 1, further comprising L-theanine in an amount of from about 100 mg to about 400 mg per serving.

8. The composition of claim 7, wherein the weight ratio of paraxanthine to L-theanine is from about 1:0.5 to about 1:1.5.

9. The composition of claim 8, wherein the weight ratio of paraxanthine to L-theanine is about 3:2.

10. The composition of claim 1, further comprising Panax ginseng root extract standardized to at least 20% ginsenosides by weight, present in an amount of from about 50 mg to about 400 mg per serving.

11. The composition of claim 1, further comprising:
(a) ascorbic acid (Vitamin C) in an amount of from about 45 mg to about 200 mg per serving;
(b) pyridoxine (Vitamin B6) in an amount of from about 1 mg to about 10 mg per serving; and
(c) methylcobalamin (Vitamin B12) in an amount of from about 2 mcg to about 50 mcg per serving.

12. The composition of claim 1, wherein the carbonation-stable spore-forming probiotic is Bacillus coagulans GBI-30, 6086 (GanedenBC30®) at about 1 billion CFU per serving.

13. The composition of claim 12, wherein the Bacillus coagulans endospores maintain at least 90% viability after 6 months storage in the carbonated aqueous base at ambient temperature and a pH of from about 2.5 to about 4.0.

14. The composition of claim 1, wherein paraxanthine is at about 300 mg, Alpha-GPC is at about 300 mg, citicoline is at about 250 mg, taurine is at about 1,000 mg, and the composition further comprises L-tyrosine at about 500 mg, L-theanine at about 200 mg, Panax ginseng root extract at about 200 mg standardized to at least 20% ginsenosides, ascorbic acid at about 90 mg, pyridoxine at about 2 mg, methylcobalamin at about 6 mcg, and Bacillus coagulans GBI-30, 6086 at about 1 billion CFU per serving.

15. The composition of claim 1, wherein the carbonated aqueous base comprises carbonated filtered water, citric acid, natural flavors, at least one zero-calorie sweetener selected from steviol glycosides, monk fruit extract, or a combination thereof, and a preservative.

16. The composition of claim 15, wherein the beverage has a pH of from about 2.5 to about 4.0 and a carbonation level of from about 3.0 to about 4.5 volumes CO₂.

17. The composition of claim 1, wherein the serving volume is about 12 fluid ounces (355 mL) and the composition is packaged in a sealed aluminum can.

18. A method of producing simultaneous cognitive enhancement and gut-brain axis modulation in a human subject, comprising administering to the subject in a single oral serving:
(a) paraxanthine (1,7-dimethylxanthine) from about 100 mg to about 500 mg, wherein paraxanthine blocks adenosine A1 and A2A receptors to disinhibit presynaptic acetylcholine release and increase dopaminergic tone;
(b) Alpha-glycerophosphocholine (Alpha-GPC) from about 100 mg to about 600 mg, wherein Alpha-GPC undergoes intraneuronal phospholipase D hydrolysis to release free choline for choline acetyltransferase-mediated acetylcholine synthesis;
(c) citicoline (CDP-choline) from about 100 mg to about 500 mg, wherein citicoline undergoes systemic hydrolysis to cytidine, which converts to uridine and enters the Kennedy pathway to maintain neuronal membrane phosphatidylcholine biosynthesis;
(d) taurine from about 500 mg to about 2,000 mg, wherein taurine provides GABAergic modulation, calcium homeostasis, and osmoregulatory support; and
(e) a carbonation-stable spore-forming probiotic at a concentration of at least 500 million CFU, wherein the probiotic germinates in the gastrointestinal tract and produces short-chain fatty acids that modulate gut-brain axis signaling;
wherein components (a), (b), (c), (d), and (e) simultaneously address five mechanistically distinct axes of cognitive and physiological modulation.

19. The method of claim 18, wherein the single oral serving is formulated as a ready-to-drink carbonated beverage of about 12 fluid ounces (355 mL).

20. The method of claim 18, wherein the single oral serving further comprises L-tyrosine in an amount of from about 200 mg to about 1,000 mg, wherein L-tyrosine serves as a substrate for tyrosine hydroxylase-mediated dopamine synthesis, providing upstream catecholaminergic substrate support complementary to paraxanthine's downstream A2A-mediated dopaminergic disinhibition.

21. The method of claim 18, wherein the single oral serving further comprises L-theanine from about 100 mg to about 400 mg at a paraxanthine-to-L-theanine weight ratio of from about 1:0.5 to about 1:1.5.

22. The method of claim 18, wherein the composition is substantially free of caffeine, theobromine, and theophylline, and administration of the composition improves cognitive function in the subject characterized by enhanced alertness, sustained working memory encoding, and absence of post-peak cognitive decline.

23. The method of claim 22, wherein paraxanthine is at about 300 mg, Alpha-GPC at about 300 mg, citicoline at about 250 mg, taurine at about 1,000 mg, the composition further comprises L-tyrosine at about 500 mg, L-theanine at about 200 mg, Panax ginseng root extract at about 200 mg standardized to at least 20% ginsenosides, ascorbic acid at about 90 mg, pyridoxine at about 2 mg, methylcobalamin at about 6 mcg, and Bacillus coagulans GBI-30, 6086 at about 1 billion CFU; the composition is formulated as a zero-sugar ready-to-drink carbonated beverage of about 12 fluid ounces; and administration comprises oral consumption of the entire serving.

24. A method of delivering a carbonation-stable probiotic in combination with a paraxanthine-based cognitive enhancement composition, comprising:
formulating a ready-to-drink carbonated beverage comprising paraxanthine from about 100 mg to about 500 mg, Alpha-glycerophosphocholine from about 100 mg to about 600 mg, citicoline from about 100 mg to about 500 mg, and Bacillus coagulans endospores at a concentration of at least 500 million CFU per serving in a carbonated aqueous base at a pH of from about 2.5 to about 4.0;
wherein the Bacillus coagulans endospores maintain at least 90% viability over a period of at least 6 months at ambient storage temperature in the carbonated aqueous base.

25. The method of claim 24, further comprising taurine from about 500 mg to about 2,000 mg and L-tyrosine from about 200 mg to about 1,000 mg per serving.

26. A ready-to-drink carbonated beverage composition comprising:
(a) paraxanthine (1,7-dimethylxanthine) from about 100 mg to about 500 mg per serving;
(b) Alpha-glycerophosphocholine (Alpha-GPC) from about 100 mg to about 600 mg per serving;
(c) citicoline (CDP-choline) from about 100 mg to about 500 mg per serving;
(d) L-tyrosine from about 200 mg to about 1,000 mg per serving;
(e) taurine from about 500 mg to about 2,000 mg per serving;
(f) L-theanine from about 100 mg to about 400 mg per serving;
wherein Alpha-GPC and citicoline are simultaneously present in the same single serving, the composition is dissolved in a carbonated aqueous base, and the composition is substantially free of caffeine, theobromine, and theophylline.

27. The composition of claim 26, further comprising a carbonation-stable spore-forming probiotic at a concentration of at least 500 million CFU per serving, wherein the probiotic is Bacillus coagulans GBI-30, 6086 in endospore form, and wherein the endospores maintain at least 90% viability after 6 months storage in the carbonated aqueous base at ambient temperature.
